(19) United States
(12) Patent Application Publication
(10) Pub. No.: US 2024/0052275 A1
(43) Pub. Date: Feb. 15, 2024

(54) SOAP-INFUSED COMPRESSED SPONGE

(71) Applicant: Esther Herzog, New York, NY (US)

(72) Inventor: Esther Herzog, New York, NY (US)

(21) Appl. No.: 18/240,639

(22) Filed: Aug. 31, 2023

Related U.S. Application Data
(63) Continuation-in-part of application No. PCT/US23/67790, filed on Jun. 1, 2023.
(60) Provisional application No. 63/396,728, filed on Aug. 10, 2022.

(51) Int. Cl. C11D 17/04; C11D 3/50; A61Q 19/10; A61K 8/02

(52) U.S. Cl. CPC C11D 17/049; C11D 3/505; A61Q 19/10; A61K 8/0216

(57) ABSTRACT

A product is provided, comprising: 1) a compressed sponge comprising a height, a length, and a width; and 2) a soap that is dried onto and/or into the compressed sponge; wherein the soap and the compressed sponge are configured such that when the product is placed under a stream of water, the soap is rehydrated and the height of the compressed sponge expands by about three times. In some embodiments, the height is about ⅓ inch when compressed and about one inch when the sponge is expanded. In some embodiments, the width is about two inches when compressed or when expanded, and wherein the length is approximately three inches when compressed or when expanded. In various embodiments, the compressed sponge may comprise a variety of materials, fragrances, types of soap, shapes, colors, and may packaged for single use or for reuse using a variety of packaging materials.

### CROSS REFERENCE TO RELATED APPLICATION

This application claims the benefit of PCT International Patent Application No. PCT/US23/67790, filed Jun. 1, 2023, which claims the benefit of U.S. Provisional Patent Application No. 63/396,728, filed Aug. 10, 2022, each of which are incorporated herein by reference.

### FIELD

The present invention relates to portable sponges and liquid soaps for use by people while traveling. It can also be used at home, in hotels, or by the elderly and disabled.

### BACKGROUND OF THE INVENTION

Traditionally, people pack both their favorite liquid soap and sponge before embarking on travel. Once in their hotel room, they then put the liquid soap into the sponge, as they would do at home. If their favorite soap is a bar, then it must be brought in a case, which isn't disposable and is inconvenient. However, packaging a full bottle of liquid uses a lot of room in one's suitcase or travel bag, plus its weight is an inconvenience.

The present invention addresses the aforementioned problems and is not described in the prior art.

### SUMMARY OF THE INVENTION

A product is provided, comprising: 1) a compressed sponge comprising a height, a length, and a width; and 2) a soap that is dried onto and/or into the compressed sponge; wherein the soap and the compressed sponge are configured such that when the product is placed under a stream of water, the soap is rehydrated and the height of the compressed sponge expands by about three times. In some embodiments, the height is about ⅓ inch when compressed and about one inch when the sponge is expanded. In some embodiments, the width is about two inches when compressed or when expanded, and wherein the length is approximately three inches when compressed or when expanded.

In some embodiments, the compressed sponge comprises one or more biodegradable materials. In some embodiments, the compressed sponge comprises polyether polyurethane, Ammonium Laureth Sulfate, Ammonium Lauryl Sulfate, Sodium Laureth-40 Sulfate, Cocamidopropyl Betaine, glycerin, Cocamide DEA, Polyquaternium-7, Sodium chloride, Polydimethylsiloxane, Ethylene glycol stearate, Allantoin, Panthenol, Citric acid, DMDM hydantoin, Hypromellose, Methyl isothiazolinone, Magnesium Chloride, and/or Magnesium Nitrate.

In some embodiments, the compressed sponge comprises a fragrance, wherein the fragrance comprises one or more volatile fragrance raw materials comprising aldehydes with a relative molecular mass of less than or equal to about 200, esters with a relative molecular mass of less than or equal to about 225, terpenes with a relative molecular mass of less than or equal to about 200, alcohols with a relative molecular mass of less than or equal to about 200 ketones with a relative molecular mass of less than or equal to about 200, nitriles, and/or pyrazines.

In some embodiments, the soap comprises hard soap, soft soap, liquid soap, dry soap, powder soap, gel soap, moisturizing soap, antibacterial soap, antifungal soap, natural soap, chemical-free soap, laundry soap, toilet soap, dish soap, bathing soap, beauty soap, medicated soap, shaving soap, or hand soap. In some embodiments, the soap comprises animal fat, vegetable oil, lye, distilled water, an essential oil, a colorant, and/or a preservative.

In some embodiments, wherein the compressed sponge is packaged for retail sale, shipping, and/or travel, and wherein the compressed sponge is individually wrapped, multiply wrapped, or wrapped in bulk.

In some embodiments, the compressed sponge is packaged in any type of recycled or unrecycled packaging material, such as plastic, paper, cardboard, foil, foam, and/or Styrofoam. In some embodiments, the compressed sponge is packaged to be disposed of after a single use. In some embodiments, the compressed sponge is configured for reuse.

In some embodiments, the compressed sponge is configured for use as a bath sponge, face sponge, pet sponge, dish sponge, car sponge, shoe sponge, cleaning sponge, or general sponge.

In some embodiments, the compressed sponge comprises a shape, wherein the shape is round, oval, square, rectangular, triangular, trapezoidal, polygonal, or irregular. In some embodiments, the compressed sponge is red, orange, yellow, green, blue, indigo, violet, brown, white, black, or combinations thereof. In some embodiments, one or more images, logos, or the like are printed on the compressed sponge.

In some embodiments, the product is safe for use by or with a variety of users comprising elderly, babies, children, adults, individuals of any gender, and/or pets.

Also provided is a process for making any of the products described herein, comprising:
a) compressing an uncompressed sponge, thereby producing the compressed sponge; and
b) drying the soap onto and/or into the compressed sponge;
   wherein the compressed sponge is configured to expand by about three times when placed under a stream of water.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 depicts the invention as packaged and sold, as viewed from above with the invention laying flat.

FIG. 2 depicts a perspective view of the invention when fully compressed, as viewed from above with the invention laying flat and removed from the packaging.

FIG. 3 depicts the invention in use. Note, it is no longer compressed, but expanded.

### DETAILED DESCRIPTION OF THE PREFERRED EMBODIMENTS

For a more complete understanding of the present invention, the objects and advantages thereof, reference is now made to the ensuing descriptions.

The present invention is a compressed sponge infused with liquid soap. Once removed from its packaging and no longer compressed, and placed under a stream of water, the invention gradually expands to a large sponge.

The present invention overcomes the difficulty of transporting a full bottle of liquid soap along with a sponge while traveling. Instead, a person need only take the invention to enjoy the use of a sponge and liquid soap while traveling.

The invention is compressed, lightweight and disposable. These attributes make it very convenient for travel.

With a little bit of water, the invention becomes an all-in-one shower companion.

### Sponge Characteristics

The compressed sponge of the present invention may be any size (i.e., any height, width, and/or length) when compressed or when expanded. In one embodiment, the height is approximately ⅓ inch when compressed and approximately one inch when the sponge is expanded. In another embodiment, the width is approximately two inches when compressed or when expanded, and the length is approximately three inches when compressed or when expanded.

The compressed sponge of the present invention may be any shape, such as round, oval, square, rectangular, triangular, trapezoidal, polygonal, or irregular.

The compressed sponge of the present invention may be made of a variety of materials. In one embodiment, the compressed sponge is made of one or more biodegradable materials. In another embodiment, the compressed sponge is made from polyether polyurethane, Ammonium Laureth Sulfate, Ammonium Lauryl Sulfate, Sodium Laureth-40 Sulfate, Cocamidopropyl Betaine, glycerin, Cocamide DEA, Polyquaternium-7, Sodium chloride, Polydimethylsiloxane, Ethylene glycol stearate, Allantoin, Panthenol, Citric acid, DMDM hydantoin, Hypromellose, Methyl isothiazolinone, Magnesium Chloride, and/or Magnesium Nitrate.

The compressed sponge of the present invention may also contain a fragrance. In general, a broad range of suitable fragrance raw materials can be found in U.S. Pat. Nos. 4,145,184; 4,209,417; 4,515,705; and 4,152,272. Any fragrance raw materials, natural oils, or the like can be used within the present invention. For example, volatile fragrance raw materials useful in the present invention may be selected from, but are not limited to, aldehydes with a relative molecular mass of less than or equal to about 200, esters with a relative molecular mass of less than or equal to about 225, terpenes with a relative molecular mass of less than or equal to about 200, alcohols with a relative molecular mass of less than or equal to about 200 ketones with a relative molecular mass of less than or equal to about 200, nitriles, pyrazines, and mixtures thereof.

The compressed sponge of the present invention may also be infused with any type of soap. In various embodiments, the compressed sponge is infused with hard soap, soft soap, liquid soap, dry soap, powder soap, gel soap, moisturizing soap, antibacterial soap, antifungal soap, natural soap, chemical-free soap, laundry soap, toilet soap, dish soap, bathing soap, beauty soap, medicated soap, shaving soap, or hand soap, and the like. In additional embodiments, any of the soaps listed above may contain one or more ingredients that include animal fat, vegetable oil, lye, distilled water, an essential oil, a colorant, and/or a preservative.

The compressed sponge of the present invention may also be any color, including red, orange, yellow, green, blue, indigo, violet, brown, white, black, and combinations thereof. In some embodiments, one or more images, logos, or the like are printed on the compressed sponge.

The compressed sponge of the present invention may be packaged for retail sale, shipping, travel, and the like. In various embodiments, the compressed sponge is individually wrapped, multiply wrapped, wrapped in bulk, or the like, and the compressed sponge is packaged in any type of recycled or unrecycled packaging material, such as plastic, paper, cardboard, foil, foam, and/or Styrofoam, and the like, as well as recycled versions thereof.

In a particular embodiment, the compressed sponge of the invention is packaged to be disposed of after a single use (e.g., once all the pre-packaged liquid soap is exhausted). However, it is envisioned that the invention can be used in a daily routine, or reused (e.g., if the liquid soap is not exhausted). At that point, the sponge portion of the invention can be reused as a sponge when the soap is exhausted.

The compressed sponge of the present invention may also be utilized for a variety of uses. In various embodiments, the compressed sponge may be a bath sponge, face sponge, pet sponge, dish sponge, car sponge, shoe sponge, cleaning sponge, general sponge, and the like, and is safe for use by or with a variety of users including the elderly, babies, children, adults, individuals of any gender, pets, and the like.

In one embodiment, the process of infusing the sponge with soap refers to adding and drying a liquid soap into the compressed sponge, such that the sponge is dry when packaged and transported, while containing dried liquid soap.

### Examples

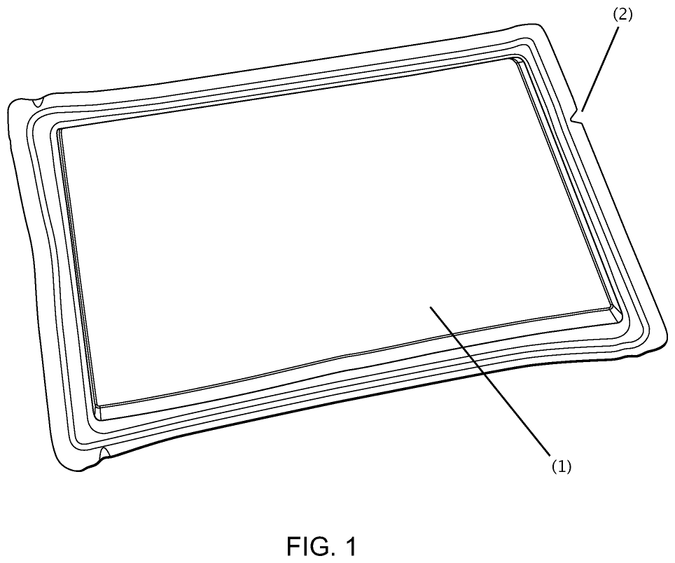

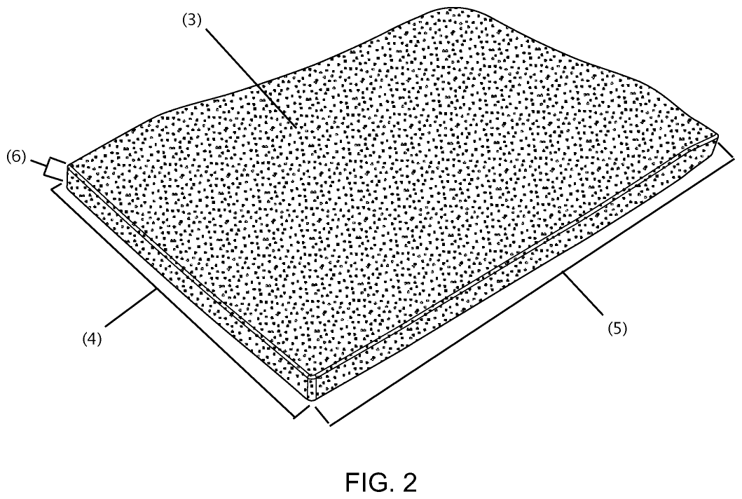

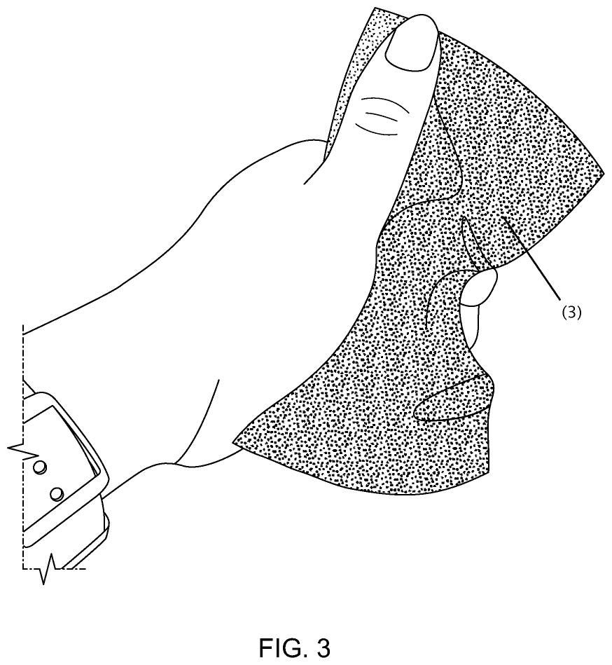

Reference is made to FIGS. 1-3, which employ a common numbering scheme.

Item (1) designates the body of the plastic covering while Item (2) designates a perforation in the plastic to assist in opening the plastic. It is this packaging that maintains the compression of the invention for transport.

Item (3) designates the body of the compressed sponge. Item (4) designates the lateral axis of the invention. Item (5) designates the horizontal axis of the invention. Item (6) designates the height axis of the invention.

In one embodiment, the lateral axis (4) is approximately two inches long. In another embodiment, the horizontal axis (5) is approximately three inches long. In another embodiment, the height axis (6) is approximately five millimeters (⅓ inch) in height when compressed. In another embodiment, the height axis is approximately one inch when the sponge is expanded.

What is claimed:

1. A product comprising:
a compressed sponge comprising a height, a length, and a width; and
a soap that is dried onto and/or into the compressed sponge;
wherein the soap and the compressed sponge are configured such that when the product is placed under a stream of water, the soap is rehydrated and the height of the compressed sponge expands by about three times.

2. The product of claim 1, wherein the height is about ⅓ inch when compressed and about one inch when the sponge is expanded.

3. The product of claim 1, wherein the width is about two inches when compressed or when expanded, and wherein the length is approximately three inches when compressed or when expanded.

4. The product of claim 1, wherein the compressed sponge comprises a shape, and wherein the shape is round, oval, square, rectangular, triangular, trapezoidal, polygonal, or irregular.

5. The product of claim 1, wherein the compressed sponge comprises one or more biodegradable materials.

6. The product of claim 1, wherein the compressed sponge comprises polyether polyurethane, Ammonium Laureth Sulfate, Ammonium Lauryl Sulfate, Sodium Laureth-40 Sulfate, Cocamidopropyl Betaine, glycerin, Cocamide DEA, Polyquaternium-7, Sodium chloride, Polydimethylsiloxane, Ethylene glycol stearate, Allantoin, Panthenol, Citric acid, DMDM hydantoin, Hypromellose, Methyl isothiazolinone, Magnesium Chloride, and/or Magnesium Nitrate.

7. The product of claim 1, wherein the compressed sponge comprises a fragrance, wherein the fragrance comprises one or more volatile fragrance raw materials comprising aldehydes with a relative molecular mass of less than or equal to about 200, esters with a relative molecular mass of less than or equal to about 225, terpenes with a relative molecular mass of less than or equal to about 200, alcohols with a relative molecular mass of less than or equal to about 200 ketones with a relative molecular mass of less than or equal to about 200, nitriles, and/or pyrazines.

8. The product of claim 1, wherein the soap comprises hard soap, soft soap, liquid soap, dry soap, powder soap, gel soap, moisturizing soap, antibacterial soap, antifungal soap, natural soap, chemical-free soap, laundry soap, toilet soap, dish soap, bathing soap, beauty soap, medicated soap, shaving soap, or hand soap.

9. The product of claim 8, wherein the soap comprises animal fat, vegetable oil, lye, distilled water, an essential oil, a colorant, and/or a preservative.

10. The product of claim 1, wherein the compressed sponge is red, orange, yellow, green, blue, indigo, violet, brown, white, black, or combinations thereof.

11. The product of claim 1, wherein one or more images and/or logos are printed on the compressed sponge.

12. The product of claim 1, wherein the compressed sponge is packaged for retail sale, shipping, and/or travel, and wherein the compressed sponge is individually wrapped, multiply wrapped, or wrapped in bulk.

13. The product of claim 12, wherein the compressed sponge is packaged in recycled or unrecycled packaging material comprising plastic, paper, cardboard, foil, foam, and/or Styrofoam.

14. The product of claim 1, wherein the compressed sponge is packaged to be disposed of after a single use.

15. The product of claim 1, wherein the compressed sponge is configured for reuse.

16. The product of claim 1, wherein the compressed sponge is configured for use as a bath sponge, face sponge, pet sponge, dish sponge, car sponge, shoe sponge, cleaning sponge, or general sponge.

17. The product of claim 1, wherein the product is safe for use by or with a variety of users comprising elderly, babies, children, adults, individuals of any gender, and/or pets.

18. A process for making the product of claim 1, comprising:
a) compressing an uncompressed sponge, thereby producing the compressed sponge; and
b) drying the soap onto and/or into the compressed sponge;
wherein the compressed sponge is configured to expand by about three times when placed under a stream of water.
